(19) United States
(12) Patent Application Publication
(10) Pub. No.: US 2016/0279444 A1
(43) Pub. Date: Sep. 29, 2016

(54) RADIOTHERAPY DOSE ASSESSMENT AND ADAPTION USING ONLINE IMAGING

(71) Applicant: SoniTrack Systems, Inc., Menlo Park, CA (US)

(72) Inventor: Jeffrey SCHLOSSER, Menlo Park, CA (US)

(73) Assignee: SoniTrack Systems, Inc., Menlo Park, CA (US)

(21) Appl. No.: 15/173,424

(22) Filed: Jun. 3, 2016

Related U.S. Application Data
(63) Continuation of application No. PCT/US2014/068927, filed on Dec. 5, 2014.
(60) Provisional application No. 61/912,985, filed on Dec. 6, 2013.

(51) Int. Cl. A61N 5/10

(52) U.S. Cl. CPC A61N 5/1071; A61N 5/1039; A61N 5/1045; A61N 5/1049; A61N 2005/1055; A61N 2005/1058

(57) ABSTRACT

In external beam radiation therapy, a planning image (scan) of the patient is obtained prior to treatment as a basis for constructing a radiation delivery plan. However, since the planning scan is obtained prior to treatment (potentially days or weeks prior), it does not necessarily represent the state of the patient's anatomy as it presents at the time of treatment beam delivery. The potential mismatch between the patient's anatomy in the planning scan and anatomy at the time of treatment can result in dose discrepancies between the planned dose and the actual delivered dose. The methods herein describe the use of online images taken immediately before or during treatment delivery in order to predict, assess, and adapt to such discrepancies.

### CROSS-REFERENCE TO RELATED APPLICATIONS

This application is a continuation of International Application No. PCT/US2014/068927 filed Dec. 5, 2014, which claims the benefit of priority to U.S. Provisional Patent Application No. 61/912,985 filed Dec. 6, 2013, each of which is incorporated herein by reference in its entirety.

### FIELD OF THE INVENTION

The present invention relates to methods and apparatus for monitoring, predicting, and adapting radiation doses based on imaging patients immediately prior to and/or during radiation beam delivery.

### BACKGROUND OF THE INVENTION

External Beam Radiation Therapy (EBRT) is used to treat more than half of all cancer patients worldwide. Traditionally in EBRT, a planning image (scan) of the patient (usually a CT or MRI image) is obtained prior to treatment as a basis for constructing a radiation delivery plan including beam angles, shapes, and intensities. The delivery plan is simulated using the information in the planning scan in order to verify that proper dosimetric criteria are met for the target and other structures within the body. However, since the planning scan is obtained prior to treatment, (potentially days or weeks prior), it does not necessarily represent the state of the patient's anatomy as it presents at the time of treatment beam delivery.

The potential mismatch between the patient's anatomy in the planning scan and anatomy at the time of treatment can result in dose discrepancies between the planned dose and the actual delivered dose. Existing systems for imaging patients prior to and during beam delivery are not able to predict, assess, and adapt to such discrepancies. The methods herein describe the use of generalized online images in order to provide this functionality.

### SUMMARY OF THE INVENTION

In treating patients with radiotherapy, methods are described for utilizing information from online imaging scans as well as planning scans. The online imaging scans may be collected before and/or during radiation therapy beam delivery in order to assess and adapt radiation dose delivered to the patient. The online images capture the state of the patient's anatomy directly prior to or during radiation beam delivery and these online images may be used to inform deformations to the planning scans that were originally used to plan and simulate the radiation dose delivered to the patient.

The deformed planning scans can then be used to compute radiation delivered to the patient in a manner that better represents the state of the patient's actual anatomy during beam delivery. While radiotherapy treatment is described, such methods are not limited to radiotherapy but can utilize a number of other medical therapies where the treatment dose can be planned and assessed, including but not limited to, high intensity focused ultrasound therapy (HIFU), radiofrequency ablations, hypothermic therapies, hyperthermic therapies, etc.

One method for estimating dose delivered during medical therapy delivery may comprise acquiring one or more planning scans of a portion of a patient body prior to medical therapy delivery; acquiring one or more online images of the portion of the patient body or in proximity to the portion prior to or during medical therapy delivery; deforming the one or more planning scans in accordance with a presentation of the one or more online images to create one or more deformed planning scans; and estimating a dose for delivery to the portion of the patient body during the medical therapy delivery using the one or more deformed planning scans. The one or more online images do riot need to align directly with or correspond to the one of more planning scans; however, there is desirably some nominal overlap between the online images and the planning scans to allow for some correspondence between the online images and scans.

Another method for assessing anatomy positions prior to, during, or subsequent to medical therapy delivery may comprise acquiring one or more planning scans of a portion of a patient body prior to medical therapy delivery; acquiring one or more online images of the portion of the patient body or in proximity to the portion prior to or during medical therapy delivery; and computing an anatomical deviation between features or structures in the one or more planning scans and the one or more online images.

Yet another method for adapting medical therapy delivery to anatomy presentation at a time of treatment may comprise acquiring one or more planning scans of a patient prior to medical therapy delivery; acquiring one or more online images of the portion of the patient body or in proximity to the portion prior to or during medical therapy delivery; deforming the one or more planning scans in accordance with a presentation of the one or more online images to create one or more deformed planning scans; and adapting a dose delivered to the patient during medical therapy delivery using the one or more deformed planning scans.

### BRIEF DESCRIPTION OF THE DRAWINGS

FIG. 1 illustrates one possible method for producing a set of deformed planning scans by registering a set of online images to a planning scan.

FIG. 2 illustrates one possible method for producing a set of deformed planning scans by using a common set of features in a collection of online images and a planning scan.

FIG. 3 illustrates one possible method for producing a set of deformed planning scans by registering online images to multiple planning scans and assessing deformation magnitudes.

FIG. 4 illustrates one possible method for producing a set of deformed planning scans by registering online images to planning scans according to motion phase.

FIG. 5 illustrates one possible method for producing a set of deformed planning scans by registering one online image to a planning scan and registering other online images to the first said online image.

FIG. 6 illustrates one possible method for producing a dose volume histogram (DVH) and dose distribution by synchronizing beams and deformed planning scans and simulating radiation delivery.

FIG. 7 illustrates one possible method for producing a dose volume histogram (DVH) by superimposing deformed planning scans on a previously calculated dose distribution.

FIG. 8 illustrates one possible method for visualizing accumulated dose computed with deformed planning scans and with the original planning scan.

FIG. 9 illustrates schematically the effect of different radiation margin strategies on target and healthy tissue dosing, highlighting the advantages of adaptive margins.

### DETAILED DESCRIPTION OF THE INVENTION

The methods described herein use information from online imaging scans collected before and/or during radiation therapy beam delivery in order to assess and adapt radiation dose delivered to the patient. The online images capture the state of the patient's anatomy directly prior to or during radiation beam delivery. The premise is to use the online images to inform deformations to the planning scans that were originally used to plan and simulate the radiation dose delivered to the patient. The deformed planning scans can then be used to compute radiation delivered to the patient in a mariner that better represents the state of the patient's actual anatomy during beam delivery. Note that while the methods below are discussed in the context of radiotherapy, it is also possible to apply such methods to other areas of medical therapy where dose can be planned and assessed including but not limited to high intensity focused ultrasound therapy (HIFU), radiofrequency ablations, hypothermic therapies, hyperthermic therapies, etc.

Online images generally refer to images of patient anatomy taken directly prior to or during radiation beam delivery. Examples of online images may include but are not limited to Positron Emission Tomography (PET) images, Single Photon Emission Computed Tomography (SPECT) images, x-ray computed tomography (CT) images, cone beam CT (CBCT) images, projection x-ray images, stereo x-ray images, external surface images, optical coherence tomography (OCT) images, photoacoustic images, magnetic resonance (MR) images or preferably, ultrasound (US) images. Online images can be nD, 1D, 2D, 3D, or 4D (real-time 3D images). In one relevant scenario, 4D US images of a tumor and/or surrounding structures are acquired by placing a probe against the patient's skin. The US probe may be held against the patient using a static fixture, mechanical arm, or robotic arm. The US images are acquired directly prior to and throughout radiation beam delivery.

Planning images (scans) generally refer to any medical images that are used to plan and simulate the radiation dose delivered to the patient. The planning scan can be a CT scan, 4DCT scan, cone beam CT scan (CBCT), MR scan, PET scan any other type of volumetric medical scan of the patient's body, or any combination of scans thereof. Note that in all methods described below, any number of intermediate images can be used to deform the planning scan based on the online images. In other words, the online images and planning scans do not necessarily need to be directly registered together, as long as the result is a deformed planning scan that maybe used to plan and simulate radiation dose delivered to the patient. For example, if the online image modality is US and the planning scan is a CT image, it could be advantageous to register the online US images to a previously acquired MR scan of the patient, and then register the MR scan to the planning CT scan to produce a deformed CT planning scan. As another example, if the online image modality is US and the planning scan is a MR image, the online US images could be registered to the MR planning scans to produce deformed MR scans. However, since MR imaging does not directly produce a tissue density map, the MR image may subsequently go through a conversion process to produce a density-based image useful for radiotherapy planning. In both cases, the end result is a deformed scan useful for radiotherapy planning, but the online image was not registered directly to the scan used for radiotherapy planning.

Throughout this document, the word “deformation” refers to a process of displacing the voxels or pixels within an image in a generalized way. The vector displacement of each voxel in the image from initial position to final “deformed” position can be represented by a vector field known as a deformation map. The word “deformable” does not imply that the relative spacing between image voxels is changed. In other words, throughout this document. “rigid” voxel displacements are included within the generalized definition of “deformable” displacements in the context of image registration, mapping, and transformation. For example, rigid translation of image voxels, rigid rotation of voxels about a fixed axis, rigid translation+rotation, scaling, and affine transformation (translation+rotation+scaling) are all valid image “deformations”.

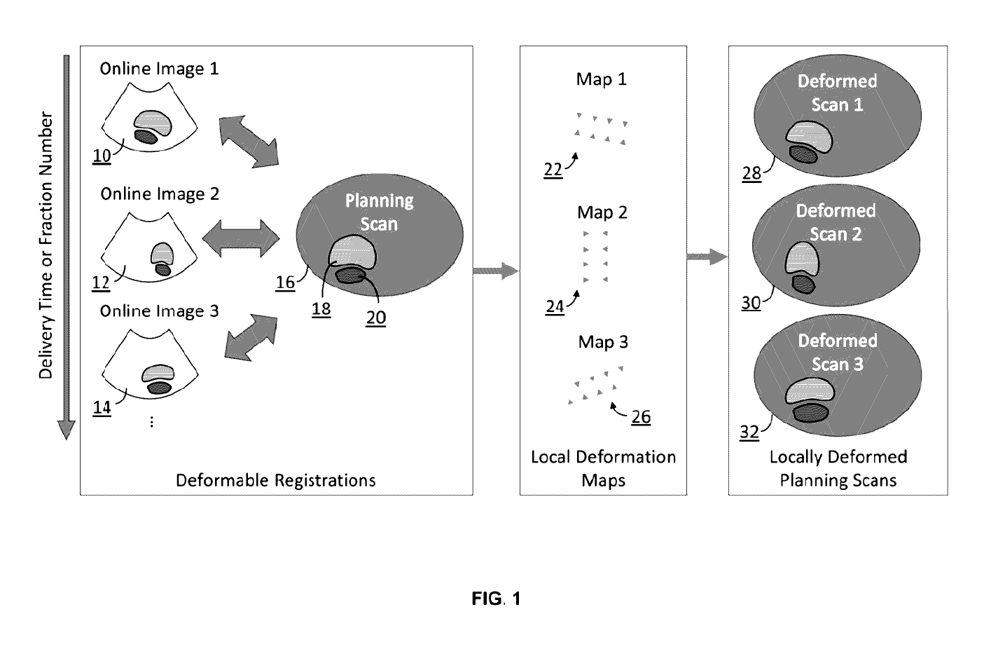

FIGS. 1, 2, 3, 4, and 5 depict several possible alternative methods of producing the deformed planning scans using one or more online images and one or more baseline planning scans. It is important to note that when planning scans and online images are registered together, the resulting deformation map is applied to deform the planning scan(s) and not the online image. The planning scan(s) contain all of the tissue density information required to compute radiotherapy dose, and in general, online images do not contain this information. Furthermore, if the online image has a restricted field of view, it may not contain sufficient anatomical information to compute dose delivery from all beam angles. The planning scan(s) by definition contain the information required to plan and compute dose delivered, and hence the planning scan(s) are deformed and used to recomputed dose delivered to the patient.

In FIG. 1, one or more online images 10, 12, 14 are registered to a single planning scan image 16 that could contain the treatment target 18 (e.g. tumor) and other relevant structures 20 (e.g. organs at risk). The result of the registrations is a set of corresponding deformation map(s) 22, 24, 26 that represent the variations in anatomy between the planning scan and online image(s). The deformation map(s) are then applied to the original planning scan in order to produce a set of deformed planning scan(s) 28, 30, 32 that match each of the online image(s).

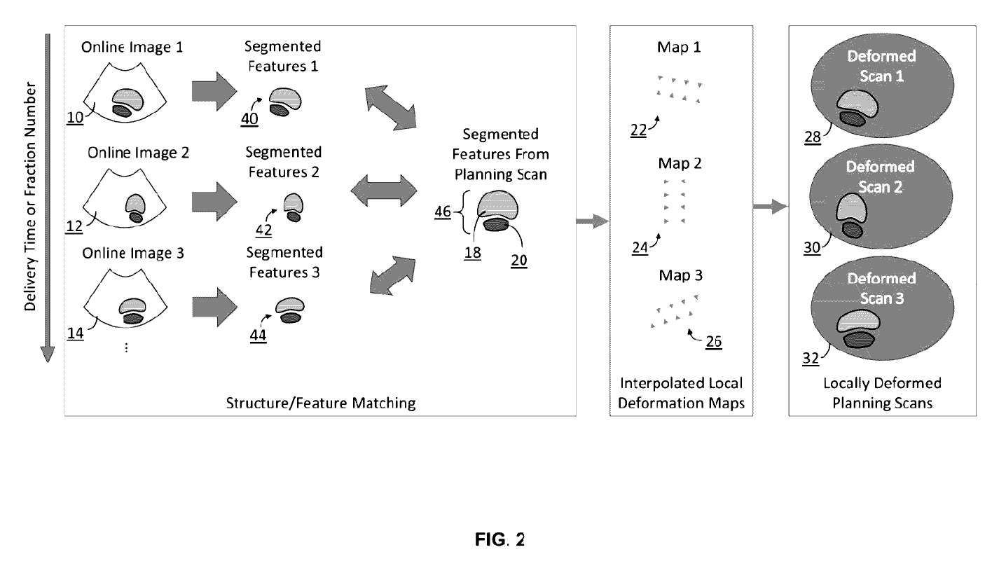

In FIG. 2, a set of specific features or structures 40, 42, 44 is identified or segmented directly within the online images 10, 12, 14 and within the planning scan(s) 46. The features or structures could include any feature or structure that can be identified in both the online images and the planning scan. Examples could be the treatment targets, gross tumor volume (GTV), surrounding structures that are segmented in the planning scan, or other high-contrast features identifiable in the online images and planning scan such as blood vessels, bone, tissue boundaries, implanted markers, skin surfaces, external markers on the surface of the patient, etc. Once a common set of features or structures is selected, displacement vectors are computed between these key structures/features in the online images and planning scan. A set of local deformation maps 22, 24, 26 is produced by interpolating and/or extrapolating the set of displacement vectors over the local region of interest. The interpolated/extrapolated local deformation maps are then applied to the planning scan to produce a set of deformed planning scans 28, 30, 32. In one possible scenario, if the online imaging modality is stereo x-ray imaging, a set of N implanted metal markers can be imaged and segmented within the stereo x-ray images and planning scan, then used to generate a set of N displacement vectors between planning scan and stereo x-ray markers. An interpolated deformation map between the planning scan and x-ray images can be generated for the local region around the target by interpolating and extrapolating the displacement of the N implanted markers to the local region surrounding the markers.

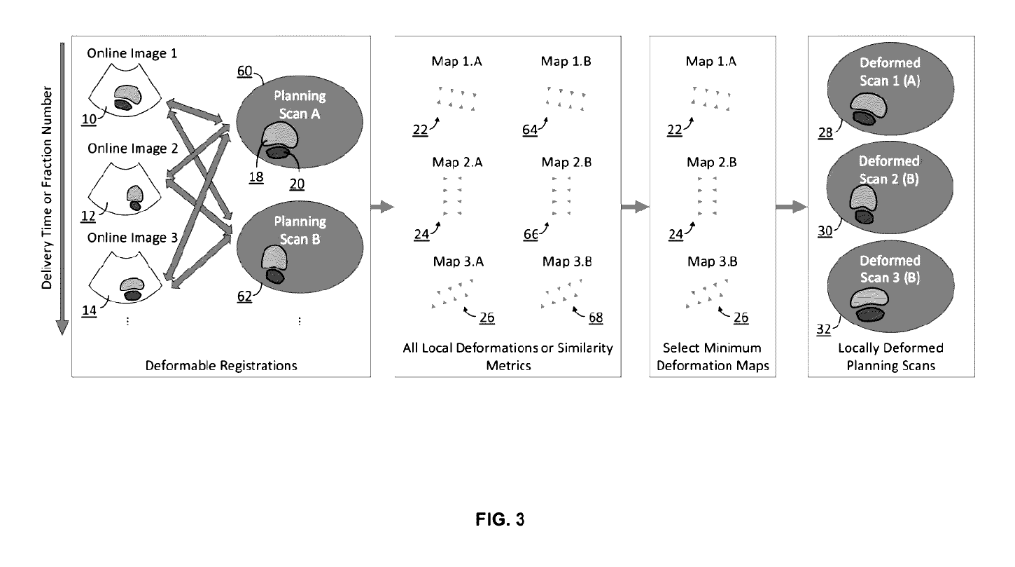

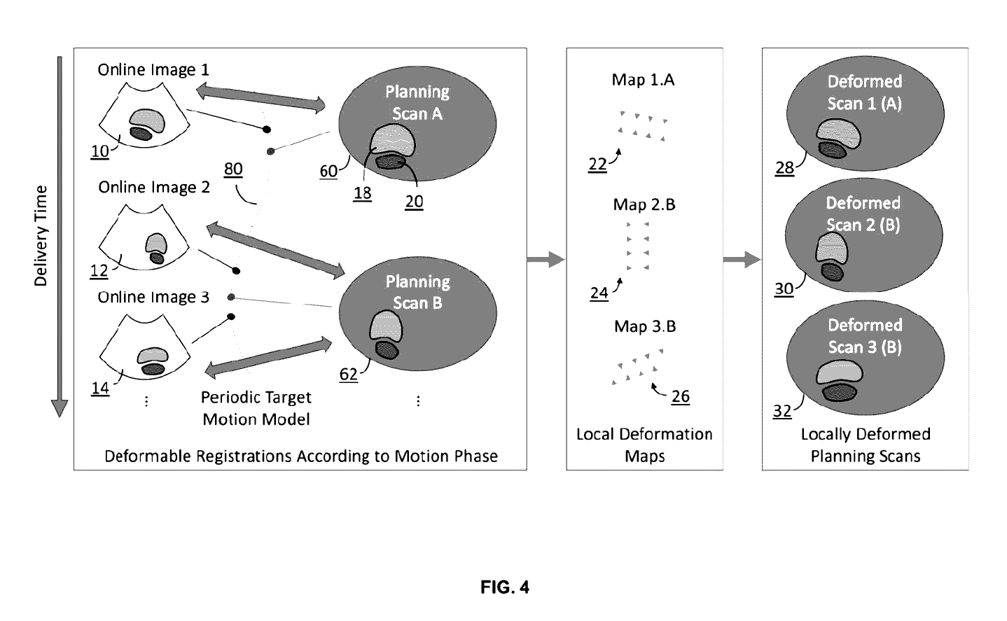

In FIGS. 3 and 4, one or more online images 10, 12, 14 are registered to multiple planning scan images 60, 62. Multiple planning scan images 60, 62 are commonly acquired in succession (e.g. using a 4D CT acquisition) when the target undergoes large periodic motions (e.g. due to breathing). Normally the planning scans are acquired at multiple points in the target's periodic motion and are used to construct, a radiation delivery plan that accounts for the target motion (for example, beam gating or beam steering). In the case of FIG. 3, if multiple planning scans are acquired, each online image can be registered to all of the planning scans. The planning scan that most closely resembles the online image is chosen as a baseline for the deformed scan corresponding to that online image. For example, online image 2 12 in FIG. 3 most closely resembles planning scan B 62, so deformed scan 2 30 uses planning scan B 62 as the baseline planning image. Deformation map 2.B 66 is used to deform the baseline planning image B 62. One way to determine resemblance between online images and planning scans is by evaluating a similarity metric between the images such as mutual information. Another way to determine resemblance is to evaluate the magnitude of the deformation maps 22, 24, 26, 64, 66, 68 resulting from registration to each planning scan image. In this case, the map with the minimum overall deformation is chosen (across all planning scans) and that corresponding planning scan is used as the baseline for subsequent deformation.

In the case of FIG. 4, each online image 10, 12, 14 is registered to the particular planning scan or scans 60, 62 that are acquired at a motion phase that is close to the motion phase at which the online image was acquired. In practice, this can be accomplished by automatically or manually tracking target motion in a sequence of planning scans 60, 62 and plotting a motion trajectory for the target. Multiple planning images can be acquired within a single period of motion in order to adequately sample and model the motion trajectory. A motion model 80 can then be fit to the planning scan target trajectories. Target motion can be automatically or manually tracked within the online images and fit to the same motion model (the planning scan model). Each image within the online and planning sequences can be assigned a particular phase within the modelled motion trajectory based on the model fit. For each online image, a registration is performed between the online image and the planning scan image whose motion phase is closest to the phase of the online image. The resulting deformation map is applied to the appropriate planning scan image to produce a deformed planning scan for that online image. For example, online image 2 12 in FIG. 4 is acquired at a motion phase closest to planning scan B 62, so deformed scan 2 30 uses planning scan B 62 as the baseline planning image. Deformation map 2.B 66 is used to deform the baseline planning image B 62. Alternatively, instead of registering the online image to the closest planning scan, an interpolated planning, scan can be produced between two sequential planning scans according to the phase at which the online image was acquired. The online image can then be registered to the interpolated planning image, and the interpolated planning image can be used as a baseline for the corresponding deformed scan.

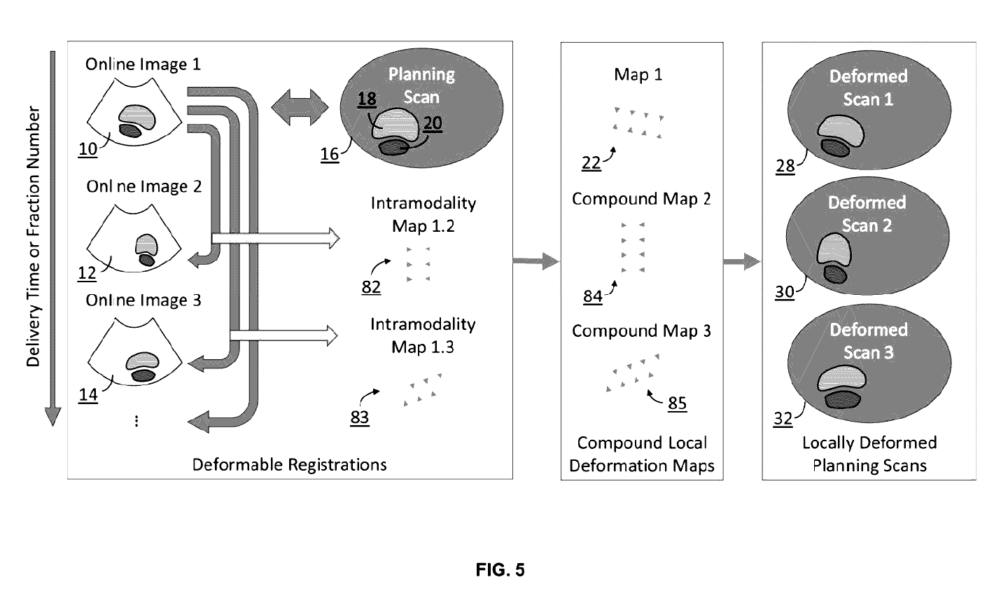

FIG. 5 depicts another alternative method for producing deformed planning scans. One online image 10 is registered to the planning scan 16, and other online images 12, 14 are registered to the first online image 10 using intramodality image registration. The deformation map 22 corresponding to online image 1 10 is the result of registration to the planning scan. The deformation maps 84, 85 are produced by first applying the deformation map 22, then applying the intramodality deformation maps 82, 83 to produce compound deformation maps 84, 85. The deformation map(s) 22, 84, 85 are then applied to the original planning scan in order to produce a set of deformed planning scan(s) 28, 30, 32 that match the corresponding online image(s).

A one-to-one relationship need not exist between online images and deformed planning scans. In other words, a set of N online images nominally yields N deformed planning scans (as shown in FIGS. 1, 2, 3, 4, 5), but can also produce less than Nor greater than N deformed planning scans. As an example when N online images could yield less than N deformed planning scans, consider a scenario where the online image modality is US and the radiotherapy target is the prostate. In this example, many intrafractional US images may be collected during beam delivery within a single fraction. If the prostate is relatively stationary throughout treatment, sequential online images may not represent significant anatomical changes, and thus a single deformed planning scan can be generated for a time period representing multiple online images. In general, a motion trigger can be employed that only generates a deformed planning scan when significant changes between sequential online images are detected. One way to implement a motion trigger is to register sequential online images together and monitor the resulting displacements or deformations. Another way to implement a motion trigger is to track the motion of particular structures within sequential images and send a trigger signal when motion exceeds a particular threshold. As an example when N online images could yield more than N deformed planning scans, consider a scenario where the online image modality is US and the radiotherapy target is the liver. In a case where US framerate is low (for example 1 volume per second), the liver target could move significantly (for example, greater than 1 cm) between US acquisitions. In this case, a deformed planning scan could be generated for every sequential US image, but in order to smoothly capture liver motion for dose calculation, additional deformed planning scans could be generated between US images. In general, additional deformed planning scans could be generated by interpolating the deformed planning scans generated directly from online images, interpolating the online images and generating deformed planning scans based on interpolated online images, or other means. Interpolation could be facilitated by using a motion model generated from the original planning scans or online images (see FIG. 4).

In certain cases, the field of view of the online image(s) is not the same as the field of view of the planning scan(s). In these cases, the deformable image registration can be performed over the field of view that is common between the online image and planning image, and the resulting deformation maps primarily encompass this shared area. For example, if the online image(s) are US images and the planning scan(s) are CT images, the US field of view is generally smaller than the CT field of view. The deformation map from the CT/US registration may primarily encompass the field of view of the US image, and hence deformation of the CT planning scan is mostly restricted to the area of the online US image (local deformation). Alternatively, the deformation map between online images and planning images can be primarily bounded by the region of the GTV, PTV, or CTV. Alternatively, the deformation map between online images and planning images can by primarily bounded by a region that includes images features commonly identified in both the online image and planning image.

In certain cases, rigid anatomy may be identified in the planning scan(s) and online image(s) that can provide constraints on non-rigid deformable registrations. For example, if the therapy target is the prostate, pelvic bony anatomy can be visible in planning CT scans and in online US images. When registering planning CTs with US images, it is known that the pelvic bony anatomy is not deformable between planning and treatment sessions, so the deformable registration can ensure that the distances between points on the pelvic bones remains unchanged in the resulting deformed planning scan.

In certain cases, by knowing the position and orientation of the online imaging device in the coordinate system of the linear accelerator (“LINAC”), which is typically used for beam radiation treatments, it may be possible to localize the voxels of the online image in the coordinate frame of the LINAC. Since the LINAC coordinate flame is linked to with the coordinate frame of the planning scan, the online image can be directly placed into the image space of the planning scan. For example, if the online image(s) are US images and the planning image(s) are CT images, the US can be directly overlaid onto the CT by tracking the US probe position with respect to the CT or LINAC frame and knowing the transformation between the physical US probe and the probe tracking sensor. Uncovering the transformation between the physical US probe and the probe tracking sensor is a well studied process called US spatial calibration. In this example, the US probe could be tracked with an optical tracking camera, an electromagnetic tracking device, a mechanical tracking device, or other means.

In certain cases, it may be possible to acquire a “baseline” online image concurrently with the planning scan, immediately prior to the planning scan, or immediately following the planning scan. By co-registering the planning scan and the baseline online image, subsequent deformable registrations between the planning scan and online images acquired at time of treatment can be simplified by deformably registering the online images to the baseline online image. Since the baseline online image is co-registered with the planning scan, the registration between the baseline online image and subsequent online images yields a deformation map between the online images and planning scan. The advantage of using a “baseline” registration is that intramodality image registration can be used (registration between images of the same modality). Without a baseline image, if the planning scans and online images represent different imaging modalities, the online and planning images are registered directly together in a process called intermodality image registration. Intermodality image registration can be challenging because of the different contrast mechanisms inherent in different medical imaging modalities.

In certain cases, if online images and planning scans are acquired with different image modalities, registration can be facilitated by simulating one or more online image(s) based on the presentation of the planning image(s). The online images can then be registered to the simulated image(s). In this way, images with similar appearance can be registered together, potentially increasing the quality of the image registration. For example, if the online images are US images and the planning images are CT images, a series of simulated US images can be generated using information in the planning CT image(s) and co-registered with the planning CT image(s). One or more simulated US images can be generated for each position of the US probe in the online US images. The simulated US images are then registered to the online US images to produce a deformation map between the online US images and the co-registered planning scan(s). Throughout this document, the process of registering online images and planning scans can refer to direct intermodality registration, intramodality registration facilitated by a baseline online image, intramodality registration facilitated by a simulated planning image, intramodality registration facilitated by compound deformations (FIG. 5), or any other means of producing a deformation map between an online image and planning image.

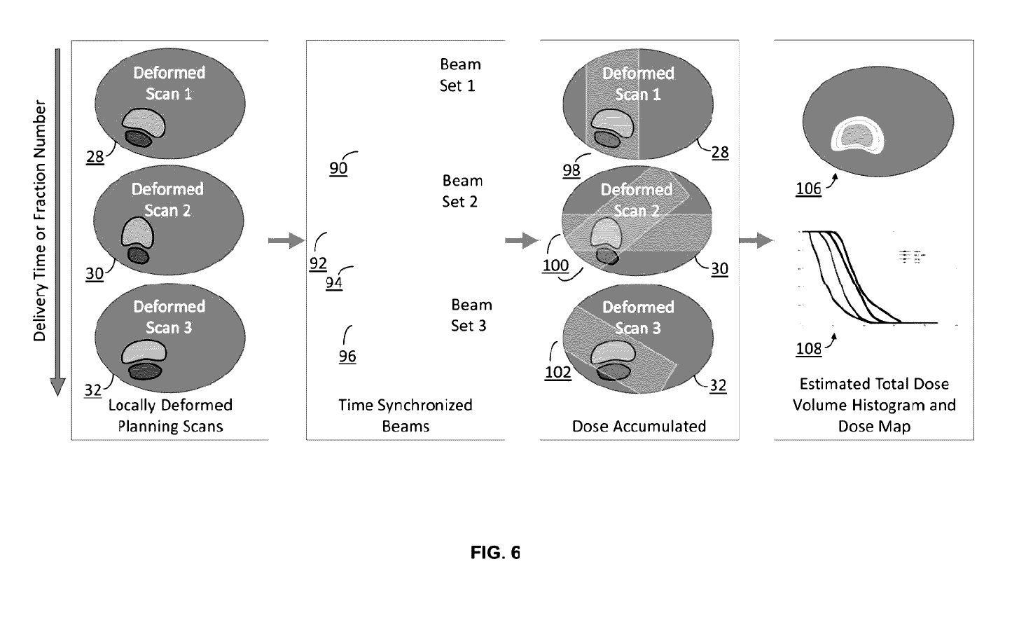

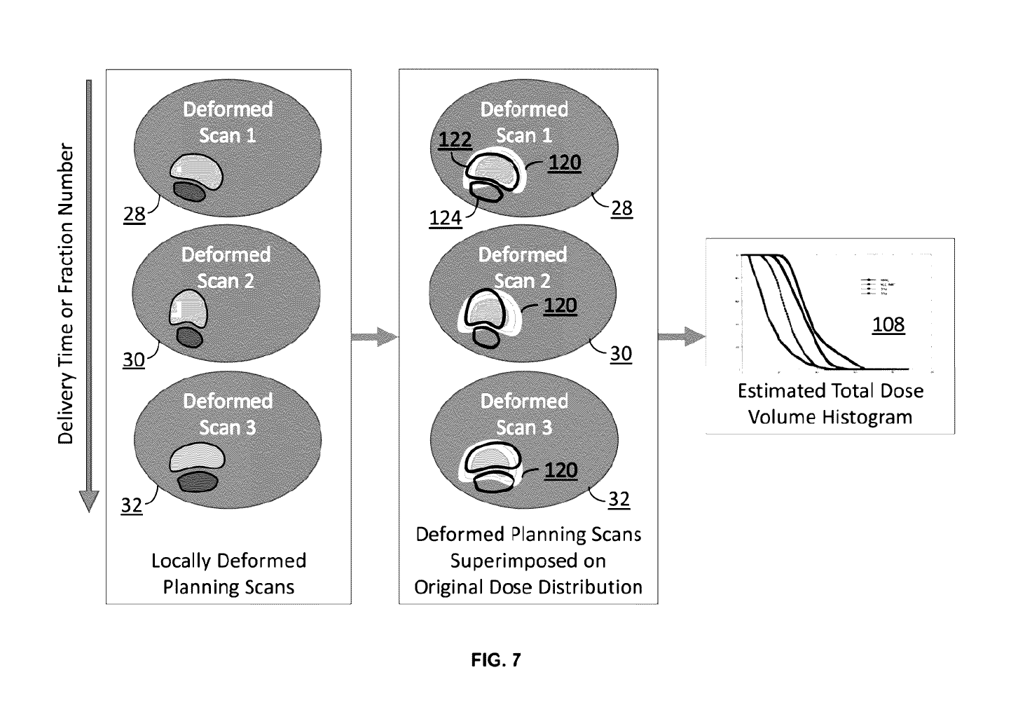

FIGS. 6 and 7 depict two alternative ways (but not the only ways) of generating dose information for radiotherapy delivery based on one or more deformed planning scans. In FIG. 6, each deformed planning scan 28, 30, 32 is synchronized to the set of beams 90, 92, 94, 96 delivered during a particular time interval. Note that either the beam plan used in the original simulation or the beams recorded by the treatment machine during actual beam delivery can be used to determine the delivered beams at a particular time during treatment. The time interval represents some interval of time over which the online image matching the deformed planning scan was acquired. The time interval can be selected as the time between the online image acquisition and the next online image acquisition, the time between the online image acquisition and the previous online image acquisition, or any variation thereof. For example, if online image 1, 2, and 3 are acquired at time 40 seconds, 50 seconds, and 60 seconds, respectively, the time interval for beams delivered to deformed planning scan 2 30 could be 45 to 55 seconds (a total of 10 seconds). If a time delay is associated with the delivery or processing of online images, the physical time of online image acquisition can be used to determine time intervals. If only one online image is acquired per fraction (e.g. directly before treatment or midway through treatment), all beams delivered for a particular fraction can be assigned to the single deformed planning scan. Dose distributions 98, 100, 102 (delivered dose) to each deformed scan 28, 30, 32 are computed by simulating delivery of the synchronized set of beams 90, 92, 94, 96 to the deformed scan(s) 28, 30, 32. A dose volume histogram (DVH) 108 can then be computed by integrating the dose delivered to each deformed set of contoured structures on the deformed planning scan(s). Furthermore, a cumulative dose distribution 106 can be displayed that sums all of the doses delivered to each deformed planning scan. The cumulative dose distribution map can be overlaid on the original planning scan or any of the deformed planning scans.

In FIG. 7, the deformed planning scans 28, 30, 32 are superimposed onto the original dose distribution map 120 computed using the original planning scan during the radiotherapy planning process. Using the original dose distribution and the superimposed deformed scans 28, 30, 32 and contoured structures 122, 124, a DVH 108 can be computed by integrating the dose delivered to each deformed set of contoured structures according to the amount of delivery time represented by each deformed scan. The amount of delivery time represents some interval of time over which the online image matching the deformed planning scan was acquired. The time interval can be selected as the time between the online image acquisition and the next online image acquisition, the time between the online image acquisition and the previous online image acquisition, or any variation thereof. For example, if online image 1, 2, and 3 are acquired at time 40 seconds, 50 seconds, and 60 seconds, respectively, the amount of delivery time for deformed planning scan 2 30 could be 10 seconds (representing the patient's anatomy state from time 45 seconds to 55 seconds). Note that when using the original dose distribution map 120 to compute the DVH 108, the original planning scan need not be fully deformed. Instead, it is possible to deform only the contoured structures relevant for computing the DVH, and overlaying those structures on the original dose distribution map.

In any embodiment, online image features (such as target and tissue boundaries) may be enhanced using contrast-enhanced imaging. This could be especially useful when tumor or surrounding tissue boundaries are not clearly visible in online images due to poor contrast. Contrast enhancement can facilitate the registration process between the online images and planning scan (FIGS. 1, 2, 3, 4, 5, or variations thereof). For example, if the online imaging modality is US and the treatment target is a liver tumor, the tumor boundaries might not be readily visible within the online US images. Contrast enhancement via microbubble injection is known to increase visibility of liver tumors, and could be used at the time of treatment to enhance tumor contrast within online images and facilitate better registration between online US images and the planning scan.

The methods described above or variations thereof can be used to estimate dose delivered to the patient after radiation delivery (interfractional dose computation). Online images acquired during treatment can be stored and used for retrospective dose computations according to the methods above. The retrospective dose computation can occur after each delivery fraction and/or after the entire treatment is completed. The methods described above or variations thereof can also be used to estimate dose delivered to the patient in real-time during delivery of a radiotherapy fraction by performing the dose computations immediately after one or more online images are acquired during radiotherapy beam delivery (intrafractional dose computation). When performing interfractional or intrafractional dose computations, estimates of the delivered dose distributions and/or DVHs can be displayed for automatic evaluation or evaluation by the radiation oncologist, therapist, or physicist.

The methods described above or variations thereof can also be used to estimate a future dose to be delivered to the patient. In one scenario, one or more online images taken directly prior to beam delivery in a given fraction can be used to predict how the deformed planning scans may present during future beam delivery. The predicted deformed planning scans can be input into the methods above (e.g. FIG. 6 and FIG. 7 or variations thereof) to predict what the resulting dose distribution or DVH may look like after beam delivery. For example, in the case of prostate radiotherapy the prostate and surrounding anatomy is typically relatively stationary throughout treatment, and hence a rough assumption is that the patient anatomy immediately prior to beam delivery is approximately the same as anatomy during beam delivery. Therefore an online image taken immediately prior to beam delivery in a given fraction can be used to generate a deformed planning scan (according to FIGS. 1, 2, 3, 4, 5, or variations thereof), and that deformed scan can be used to predict the future dose distribution or future DVH according to FIG. 6 or FIG. 7 or variations thereof As another example, in the case of liver radiotherapy, the anatomy undergoes large amplitude periodic motion. A series of online images can be taken immediately prior to beam delivery in a given fraction to sample the nature of liver motion immediately prior to treatment. These images can be used to generate a set of deformed planning scan(s) representative of one or more liver motion cycles. The set of deformed planning scans(s) can then be used to predict the future dose distribution or future DVH according to the methods above.

Interfractionat intrafractional, or predicted dose computations can be compared to the dose estimates based on the original planning scan. In one method, the original planning scan can be substituted for the deformed planning scans in the methods above (FIG. 6 and FIG. 7 or variations thereof), and the resulting DVHs or dose distributions at any treatment time can be directly compared to those generated with the intrafractional, interfractional, or predicted deformed planning scans. If meaningful dose deviations are detected interfractionally or intrafractionally, the beam delivery parameters can be redesigned to compensate for the deviations and meet the original overall dosimetric criteria. If intrafractional dose estimation or dose prediction is used, an alarm can be triggered if the dose delivered or predicted has deviated beyond a particular threshold relative to the planned dose. In one possible illustrative scenario, delivered doses are computed intrafractionally using methods above. The predicted total dose delivered to the patient at the end of the fraction or at the end of treatment is generated in real-time (using methods in FIG. 6, FIG. 7, or variations thereof) by combining the deformed planning scans based on online intrafractional imaging (FIG. 1, 2, 3, 4, 5, or variations thereof) with predicted deformed planning scans extrapolated to the end of treatment or the end of the fraction. Predicted total dose delivered is compared with the original planned total dose delivered by visualizing both dose distributions and both DVH plots. If at any time the predicted dose distribution or predicted DVH deviate beyond a certain threshold from the corresponding planned dose distribution or planned DVH, an alarm is triggered, treatment is stopped, and beams are replanned to meet the original dosimetric criteria using knowledge of the dose already delivered to the patient.

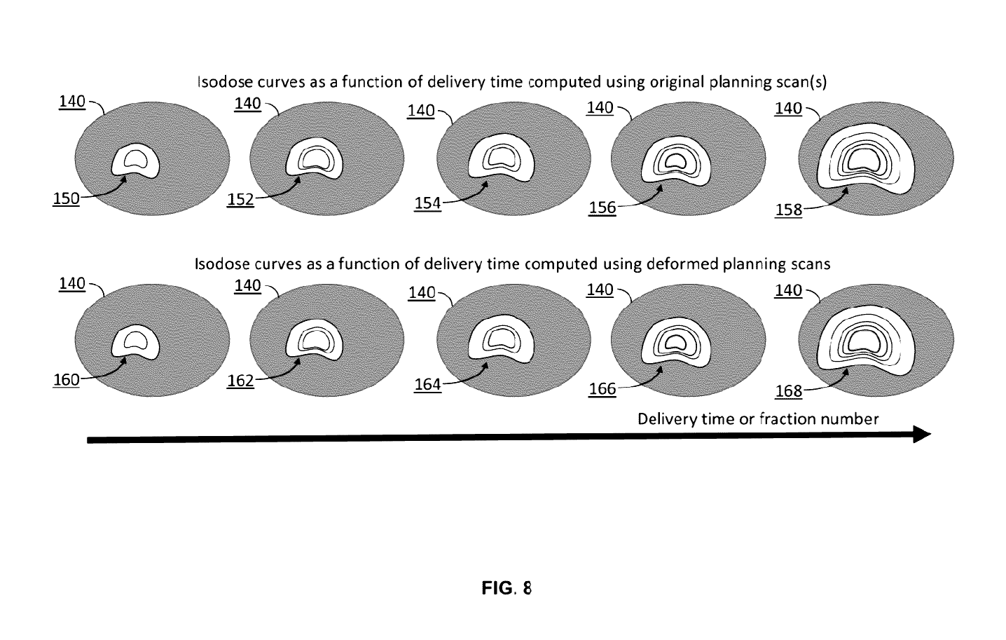

A visualization platform can be implemented to review the accumulated dose as a function of delivery time and/or fraction number. The DVHs, dose maps, and/or isodose curves can be shown and updated based on a specified time within a single fraction or within the patient's entire treatment regimen. A playback can be implemented that displays the dose accumulating as each fraction progresses, based on the real-time information extracted from the online images. An accompanying set of DVHs, dose maps, and/or isodose curves can be shown for the originally planning dose delivery. FIG. 8 shows an example of visualizing isodose curves 150, 152, 154, 156, 158, 160, 162, 164, 166, 168 overlaid on planning scans 140 as a function of delivery time or fraction number. One set 160, 162, 164, 166, 168 is computed based on a set of deformed planning scans and another set 150, 152, 154, 156, 158 is computed based on the original planning scan for comparison.

In a related method, instead of fully computing or predicting delivered dose using determined planning scans, other information can be used to assess the extent of anatomy deviation from the planning scan. If anatomy deviations exceed a particular threshold (without necessarily estimating or predicting the actual dose delivered), a cautionary flag can be triggered that questions the validity of the delivered dose (in the case the online images are acquired during beam delivery) or the treatment to be administered (in the case the online images are acquired prior to beam delivery). In other words, online imaging can be used to compare anatomical configuration or anatomical motion with expected configuration or motion. In the scenario where the target anatomy does not undergo periodic motion, deformation of the target and surrounding anatomy can be captured in online images and compared with the original planning scan. One way to perform this comparison is to deformably register the online image and the planning scan according to method above, and determine the magnitude of the deformation map. If the deformation map exceeds a particular deformation threshold (for example, maximum deformation of a certain number of millimeters or target displacement of a certain number of millimeters), a cautionary trigger signal can he activated. Another way to perform this comparison is to compare the area, volume, surface area, shape, or other attributes of the contoured structures in the original planning scan to the structures in the online images or the structures in corresponding deformed planning scans. In the scenario where the target undergoes periodic motion, motion of the target and/or surrounding structures captured or tracked within sequential online images (“online motion”) can be compared to expected motion portrayed in a set of 4D planning scans or in “baseline” online images acquired at the time of treatment planning (“planned motion”). Radiotherapy treatment margins and delivery strategies are usually designed in advance to conform to expected target trajectory (“planned motion”). If online motion deviates from planned motion more than a particular threshold, a cautionary trigger signal can be activated. Planned motion and online motion can be compared in several ways. One way is to correlate the online motion trajectory to the planned motion trajectory (for example using cross correlation) and measure the correlation coefficient. Another way is to fit a model to the planned motion, fit the online motion to the planned model, and measure the model fit. Such motion and deformation comparisons help roughly determine whether the radiation will be delivered to patient anatomy in a manner sufficiently close to the planned delivery, without fully computing/predicting the dose to be delivered using the deformed planning scan methods described above.

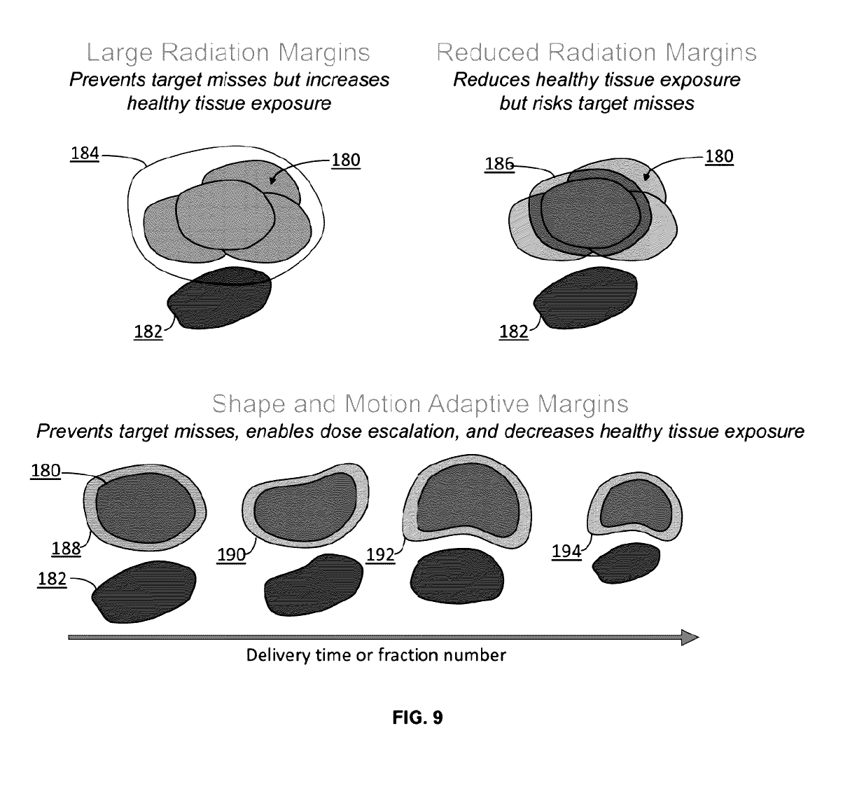

Online image information collected prior to and/or during beam delivery can be used to adapt the radiation delivery margins in real-time. FIG. 9 illustrates the clinical advantage of using radiation margins that adapt to shape, deformations, and real-time motions of the tumor/target and/or healthy organ(s). Large radiation margins 184 prevent target misses as the target changes positions during beam delivery 180, but increase healthy tissue 182 exposure. Reduced radiation margins 186 that remain fixed throughout treatment reduce healthy tissue 182 exposure but risk target misses if the target is mobile 180. Adaptive margins 188, 190, 192, 194 reduce chance of target 180 misses and target underdosing, while at the same time reducing healthy tissue 182 exposure. One of the key challenges of adaptive therapy is understanding the underlying anatomy presentation and motion at the time of treatment in order to adapt the margins appropriately. As described previously in this document, online image can be used to monitor the patient's internal anatomy and deform the planning scan (FIG. 1, 2, 3, 4, 5, or variations thereof). The resulting deformed target contour (e.g. PTV) on the planning scan can be used as the adaptive margin for therapy delivery. In one embodiment, multi-leaf collimator leaves on the linear accelerator can be instructed to adapt to the real-time updated target margin during beam delivery to account for target motions and deformations. In another embodiment, a robotic linear accelerator can be instructed to continuously compensate for target motion and deformation when irradiating the target. In another embodiment—several radiation therapy treatment plans are constructed after the patient's original planning scan. The treatment plan that best suits the online-measured anatomy position and motion before treatment (as indicated by the deformed planning contours) is selected for use during therapy. In another embodiment, new beam angles and shapes are selected immediately before treatment in accordance with the deformed anatomy contours.

Modification of the above-described assemblies and methods for carrying out the invention, combinations between different variations as practicable, and variations of aspects of the invention that are obvious to those of skill in the art are intended to be within the scope of the claim.

What is claimed is:

1. A method for estimating dose delivered during medical therapy delivery comprising:
a. acquiring one or more planning scans of a portion of a patient both prior to medical therapy delivery:
b. acquiring one or more online images of the portion of the patient body or in proximity to the portion prior to or during medical therapy delivery;
c. deforming the one or more planning scans in accordance with a presentation of the one or more online images to create one or more deformed planning scans; and
d. estimating a dose for delivery to the portion of the patient body during the medical therapy delivery using, the one or more deformed planning scans.

2. The method of claim 1 wherein acquiring the one or more online images comprises acquiring ultrasound images of the portion of the patient body.

3. The method of claim 1 wherein acquiring the one or more planning scans comprises acquiring CT or MRI images of the portion of the patient body.

4. The method of claim 1 further comprising delivering radiation therapy.

5. The method of claim 1 wherein estimating the dose comprises synchronizing the one or more deformed planning scans with beam information delivered over an interval where a matching online image was acquired.

6. The method of claim 1 wherein estimating the dose comprises using a dose map computed from the one or more planning scans.

7. The method of claim 1 wherein estimating the dose comprises retroactively estimating the dose after medical therapy delivery.

8. The method of claim 1 wherein estimating the dose comprises computing the dose during medical therapy delivery.

9. The method of claim 8 further comprising displaying the estimated dose during medical therapy delivery.

10. The method of claim 1 wherein estimating the dose comprises computing the dose before delivery of one or more medical therapy sessions.

11. The method of claim 1 further comprising comparing an estimated first dose based on the one or more deformed planning scans against an estimated second dose based on the one or more planning scans.

12. The method of claim 11 wherein comparing the estimated first dose against the estimated second dose comprises comparing a dose distribution or DVH.

13. The method of claim 11 further comprising triggering a signal if the estimated first dose estimated second dose differ beyond a threshold limit.

14. The method of claim 11 further comprising displaying the estimated dose during medical therapy delivery.

15. The method of claim 1 wherein deforming further comprises computing a deformed planning scan when a motion trigger from the one or more online images is activated.

16. A method for adapting, medical therapy delivery to anatomy presentation at a time of treatment comprising:
a. acquiring one or more planning scans of a patient prior to medical therapy delivery;
b. acquiring one or more online images of the portion of the patient body or in proximity to the portion prior to or during medical therapy delivery;
c. deforming the one or more planning scans in accordance with a presentation of the one or more online images to create one or more deformed planning scans; and
d. adapting a dose delivered to the patient during medical therapy delivery using the one or more deformed planning scans.

17. The method of claim 16 wherein acquiring the one or more online images comprises acquiring ultrasound images of the portion of the patient body.

18. The method of claim 16 wherein acquiring the one or more planning scans comprises acquiring CT or MRI images of the portion of the patient body.

19. The method of claim 16 further comprising delivering radiation therapy.

20. The method of claim 16 wherein adapting a dose comprises adjusting one or more margins for the medical therapy delivery based on a deformed presentation of contoured structures within the one or more planning scans.

21. The method of claim 20 where the one or more margins are continuously adapted during the medical therapy delivery using a multi-leaf collimator.

22. The method of claim 20 where the one or more margins are continuously adapted during the medical therapy delivery using a robotic linear accelerator.
